(19) United States
(12) Patent Application Publication
(10) Pub. No.: US 2022/0220551 A1
(43) Pub. Date: Jul. 14, 2022

(54) COMPOSITIONS AND METHODS FOR CORRECTING FOR CELLULAR ADMIXTURE IN EPIGENETIC ANALYSES

(71) Applicant: Behavioral Diagnostics, LLC, Coralville, IA (US)

(72) Inventor: Robert Philibert, Iowa City, IA (US)

(21) Appl. No.: 17/605,019

(22) PCT Filed: Apr. 22, 2020
(86) PCT No.: PCT/US2020/029266

§ 371 Date: Oct. 20, 2021

Related U.S. Application Data
(60) Provisional application No. 62/836,890, filed on Apr. 22, 2019.

(51) Int. Cl. C12Q 1/6858; C12Q 1/6881

(52) U.S. Cl. CPC C12Q 1/6858; C12Q 2600/154; C12Q 1/6881

(57) ABSTRACT

This disclosure relates to differentially methylated regions (DMRs) and an equation that can be applied when using epigenetic analysis in a biological sample that includes more than one cell type and, therefore, more than one methylation set point (e.g., saliva). This disclosure relates to differentially methylated regions (DMRs) and an equation that can be applied when using epigenetic analysis in a biological sample that includes more than one cell type and, therefore, more than one methylation set point (e.g., saliva).

### CROSS REFERENCE TO RELATED APPLICATIONS

This application claims the benefit of priority under 35 U.S.C. 119(e) to U.S. Application No. 62/836,890 filed Apr. 22, 2019.

### FEDERALLY SPONSORED RESEARCH OR DEVELOPMENT

This invention was made with government support under R44AA022041, R44DA041014, and R44CA213507 awarded by the Small Business Administration. The government has certain rights in the invention.

### TECHNICAL FIELD

This disclosure generally relates to epigenetic analysis.

### BACKGROUND

Buccal cells have been shown to be a more informative surrogate tissue than blood for epigenome-wide association studies (see, e.g., Lowe et al., 2013, Epigenetics, 8(4):445-54). However, the DNA from saliva is more difficult to analyze with respect to DNA methylation because the DNA in saliva originates from two distinct tissues, buccal cells that are sloughed from the oral laryngeal cavity and white blood cells that marginate in from the gums or salivary glands/parotid glands. Since DNA methylation set points can vary between tissues, studies of health conditions that use saliva DNA as a source for methylation data can be challenging to conduct.

### SUMMARY

This disclosure relates to differentially methylated regions (DMRs) and an equation that can be applied when using epigenetic analysis in a biological sample that includes more than one cell type and, therefore, more than one methylation set point (e.g., saliva).

In one aspect, methods of correcting for cellular heterogeneity in an oropharyngeal biological sample are provided, where the biological sample can be used to determine the methylation status of a target nucleic acid sequence. Such methods typically includes providing the oropharyngeal biological sample, the oropharyngeal biological sample comprising buccal cells and white blood cells; determining the methylation status of the target sequence and at least one differentially methylated region (DMR) loci in the biological sample; applying a formula to the methylation status of the target sequence and the at least one DMR loci in the biological sample to determine an amount of white blood cells and an amount of buccal cells in the biological sample; and correcting for cellular heterogeneity in the biological sample when determining the DNA methylation status of the target sequence.

In some embodiments, the oropharyngeal biological sample is saliva or sputum.

In some embodiments, the absolute difference between the methylation status at the DMR loci in whole blood and at the DMR loci in buccal cells is at least 0.5 (e.g., at least 0.6, at least 0.7, at least 0.8, or at least 0.9).

In some embodiments, the DMR loci is selected from DMR11 (cg25574765), DMR20 (cg03841065), DMR11 (cg10511890), DMR12 (cg08075204), DMR7 (cg24620436), DMR20 (cg07598052), DMR16 (cg04921315), DMR11 (cg26427109), DMR2 (cg00438740), DMR6 (cg09344348), DMR11 (cg08141395), DMR10 (cg24681845), DMR19 (cg22824635), DMR4 (cg14516100), and DMR1 (cg20820767).

In some embodiments, the DMR loci is DMR16 and the formula comprises

DMR16(obs)=(0.97X+0.18(1−X))

wherein DMR16(cg05575921) (obs) is the observed methylation signal in the heterogeneous biological sample; and X is the white blood cell contribution to the biological sample.

In some embodiments, the DMR loci is DMR11 and the formula comprises

DMR11(obs)=(0.01X+0.99(1−X))

wherein DMR11(cg08141395) (obs) is the observed methylation signal in the heterogeneous biological sample; and X is the white blood cell contribution to the biological sample.

In some embodiments, the determining step comprises PCR and/or sequencing.

In another aspect, methods of correcting for cellular heterogeneity in a biological sample are provided. Such methods typically include (a) providing a heterogeneous biological sample comprising buccal cells and white blood cells; (b) contacting nucleic acid from the biological sample with bisulfite under alkaline conditions; (c) performing methylation-sensitive PCR on the bisulfite-converted nucleic acid with a pair of primers that amplifies a first locus comprising at least one target CpG dinucleotide and a pair of primers that amplifies at least one DMR loci; (d) determining the methylation status of the at least one target CpG dinucleotide and the methylation status of the at least one DMR loci; and (e) correcting for cellular heterogeneity in the biological sample using a pre-determined formula.

In some embodiments, the absolute difference between the methylation status at the DMR loci in whole blood and the DMR loci in buccal cells is at least 0.5 (e.g., at least 0.6, at least 0.7, at least 0.8, or at least 0.9).

In some embodiments, the DMR is selected from DMR11 (cg25574765), DMR20 (cg03841065), DMR11 (cg10511890), DMR12 (cg08075204), DMR7 (cg24620436), DMR20 (cg07598052), DMR16 (cg04921315), DMR11 (cg26427109), DMR2 (cg00438740), DMR6 (cg09344348), DMR11 (cg08141395), DMR10 (cg24681845), DMR19 (cg22824635), DMR4 (cg14516100), and DMR1 (cg20820767).

In some embodiments, the DMR loci is DMR16 and the predetermined formula comprises

DMR16(obs)=(0.97X+0.18(1−X))

wherein DMR16(obs) is the observed methylation signal in the biological sample; and X is the white blood cell contribution to the biological sample.

In some embodiments, the DMR loci is DMR11(cg08141395) and the predetermined formula comprises

DMR11(cg08141395)(obs)=(0.01X+0.99(1−X))

wherein DMR11(cg08141395) (obs) is the observed methylation signal in the biological sample; and X is the white blood cell contribution to the biological sample.

In some embodiments, the determining step further comprises sequencing.

In still another aspect, methods for identifying a differentially methylated region (DMR) loci that can be used to correct for cellular heterogeneity in a biological sample is provided. Such methods typically include (a) comparing the methylation status of a plurality of loci in a first component of the heterogeneous biological sample and the methylation status of a plurality of loci in a second component of the heterogeneous biological sample; (b) identifying one or more loci from the plurality of loci that are differentially methylated in the first component of the heterogeneous biological sample relative to the second component of the heterogeneous biological sample, wherein the absolute difference between the methylation status in the first component and the methylation status in the second component of the one or more identified loci is at least 0.5 (e.g., at least 0.8, at least 0.9), thereby identifying a DMR loci that can be used to correct for cellular heterogeneity in a biological sample.

In some embodiments, the DMR is DMR11 (cg25574765), DMR20 (cg03841065), DMR11 (cg10511890), DMR12 (cg08075204), DMR7 (cg24620436), DMR20 (cg07598052), DMR16 (cg04921315), DMR11 (cg26427109), DMR2 (cg00438740), DMR6 (cg09344348), DMR11 (cg08141395), DMR10 (cg24681845), DMR19 (cg22824635), DMR4 (cg14516100), and DMR1 (cg20820767).

In yet another aspect, articles of manufacture are provided that can be used to correct for cellular heterogeneity in a biological sample when determining the nucleic acid methylation status of a target sequence in the biological sample. Such articles of manufacture typically include a first pair of DMR primers; and at least one DMR probe that detects either a methylated or an unmethylated CpG dinucleotide. In some embodiments, an article of manufacture further includes a second pair of DMR primers.

In some embodiments, the article of manufacture includes a first pair of DMR11 primers; and at least one DMR11 probe that detects either a methylated or an unmethylated CpG dinucleotide.

In some embodiments, the first pair of DMR11 primers includes a first member and a second member, wherein the first member has the sequence shown in SEQ ID NO:12 and the second member has the sequence shown in SEQ ID NO:15. In some embodiments, the at least one DMR11 probe is selected from the sequence shown in SEQ ID NO:16 and the sequence shown in SEQ ID NO:17.

In some embodiments, the article of manufacture further includes a second pair of DMR11 primers. In some embodiments, the second pair of DMR11 primers includes a first member and a second member, wherein the first member has the sequence shown in SEQ ID NO:13 and the second member has the sequence shown in SEQ ID NO:14.

In some embodiments, the article of manufacture includes a first pair of DMR16 primers; and at least one DMR16 probe that detects either a methylated or an unmethylated CpG dinucleotide.

In some embodiments, the first pair of DMR16 primers includes a first member and a second member, wherein the first member has the sequence shown in SEQ ID NO:3 and the second member has the sequence shown in SEQ ID NO:5. In some embodiments, the at least one DMR16 probe is selected from the sequence shown in SEQ ID NO:7 and the sequence shown in SEQ ID NO:8.

In some embodiments, the article of manufacture further includes a second pair of DMR16 primers. In some embodiments, the second pair of DMR16 primers includes a first member and a second member, wherein the first member has the sequence shown in SEQ ID NO:4 and the second member has the sequence shown in SEQ ID NO:6.

In some embodiments, at least one member of the first pair of primers, at least one member of the second pair of primers, or the at least one probe comprises a modified nucleotide (e.g., locked nucleic acid).

In some embodiments, the article of manufacture further includes reagents for bisulfite converting nucleic acid. In some embodiments, the article of manufacture further includes reagents for amplifying nucleic acid. In some embodiments, the article of manufacture further includes at least one probe that detects either the methylated or the unmethylated CpG dinucleotide. In some embodiments, the article of manufacture further includes a minor groove binder (MGB).

In one aspect, methods for detecting the methylation status of at least one CpG dinucleotide within DMR16 in a biological sample from a subject are provided. Such methods generally include (a) providing the biological sample from the subject; (b) contacting DNA from the biological sample with bisulfite under alkaline conditions; (c) contacting the bisulfite-converted DNA with a pair of oligonucleotide probes that amplifies at least one CpG dinucleotide within a differentially methylated region (DMR) of chromosome 16 (DMR16), wherein the pair of oligonucleotide probes hybridizes to and amplifies the bisulfite-converted nucleic acid sequence that comprised, prior to being contacted with the bisulfite, the at least one CpG dinucleotide in an unmethylated form; and (d) determining the methylation status of the at least one CpG dinucleotide within DMR16.

In another aspect, methods of correcting for cellular heterogeneity in a biological sample when determining the DNA methylation status of a target sequence are provided, wherein the biological sample is saliva. Such methods generally include providing the biological sample; determining the methylation status of nucleic acid in the biological sample, wherein the nucleic acid for which the methylation status is determined comprises the target sequence and a differentially methylated region of chromosome 16 (DMR16) sequence; and applying a formula to the methylation status of the DMR16 sequence to determine the relative amount of white blood cells in the total biological sample, thereby correcting for cellular heterogeneity in the biological sample when determining the DNA methylation status of the target sequence.

In one embodiment, the formula comprises DMR16(obs)=(0.97X+0.18(1−X)), wherein DMR16(obs) is the observed methylation signal in the biological sample and X is the white blood cell contribution to the biological sample.

In still another aspect, methods of using methylation-sensitive polymerase chain reaction (PCR) to correct for cellular heterogeneity in a biological sample are provided. Such methods generally include (a) providing the biological sample; (b) contacting DNA from the biological sample with bisulfite under alkaline conditions; (c) performing methylation-sensitive PCR on the bisulfite-converted DNA with a pair of oligonucleotide probes that amplifies a locus comprising at least one CpG dinucleotide, wherein the pair of oligonucleotide probes hybridizes to and amplifies the bisulfite-converted nucleic acid sequence that comprised, prior to being contacted with the bisulfite, the at least one CpG dinucleotide in an unmethylated form; and (d) determining the methylation status of the at least one CpG dinucleotide. In some embodiments, the locus is DMR16.

In another aspect, articles of manufacture are provided (that allows for the correction for cellular heterogeneity in a biological sample when determining the DNA methylation status of a target sequence), comprising: a (first) pair of primers having the sequences shown in SEQ ID NOs:3 and 5; a (second) pair of primers having the sequences shown in SEQ ID NOs:4 and 6; and at least one probe that detects an unmethylated CpG dinucleotide.

In some embodiments, the article of manufacture further includes reagents for bisulfite converting nucleic acid. In some embodiments, the article of manufacture further includes reagents for amplifying and nucleic acid. In some embodiments, the article of manufacture further includes at least one pair of primers for amplifying a target sequence comprising a CpG dinucleotide.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DESCRIPTION OF DRAWINGS

FIG. 1 is a logistic plot of the relationship of whole blood cg05575921 methylation to smoking status. The results from the smokers (n=99) are to the left of the curved line, while the results from the non-smoking subjects (n=78) are to the right of the blue curve.

FIG. 2 is a plot showing the relationship of daily cigarette consumption (cigarettes per day) as a function of methylation status.

FIG. 3 is a plot showing the relationship of pack-per-year consumption as a function of methylation status (n=346).

FIG. 4 is a plot showing the relationship of cg05575921 methylation in DNA prepared from whole blood versus cg05575921 methylation in DNA prepared from saliva (n=274).

FIG. 5 is a bar graph showing the percent contribution of whole blood DNA to the total human DNA concentration in saliva DNA (n=301).

FIG. 6 is a logistic plot of the relationship of cg05575921 methylation to smoking status in saliva DNA without correction for cellular heterogeneity. The results from the smokers (n=99) are to the left of the curved line while the results from the non-smoking subjects (n=78) are to the right of the blue curve.

FIG. 7 is data from experiments in which methylation was examined for alcohol use using saliva.

FIG. 8 is data from experiments in which methylation was examined for alcohol use using saliva and corrected for cellular heterogeneity using DMR16.

Like reference symbols in the various drawings indicate like elements.

### DETAILED DESCRIPTION

There are approximately 28 million CpG sites in the human genome, as well as tens of thousands of non-canonical methylation sites. Therefore, DNA methylation assessments are becoming increasingly accepted as methods through which to assess important health conditions such as cardiovascular disease and biological age, as well as to assess the use of, for example, nicotine, alcohol, cannabis and other drugs. Many methylation sites, however, have substantial differences in methylation status from one tissue to another, the most common example being whole blood versus buccal cells. Understanding how this correction can be done is important if any of the methylation diagnostic information obtained using blood-based DNA approaches can be used in assessments of saliva or another heterogeneous oropharyngeal biological sample such as sputum.

Most methods to date have used DNA prepared from whole blood, primarily because of its compatible with current medical procedures and the fact that DNA from whole blood is derived from a single tissue. The use of cells from a single tissue minimizes significantly, but does not completely eliminate, the effects of cellular heterogeneity on methylation values. This makes the use of blood, which is readily obtained in biomedical research and can be transformed into a source material for a wide variety of biological investigations, an ideal source of DNA for methylation studies. Still, it is not an ideal source of biomaterials for DNA methylation studies because obtaining blood in sufficient quantities typically necessitates phlebotomy and because conducting phlebotomy is time-consuming, costly and many subjects do not participate in biomedical research because of their aversion to needles.

In contrast, most research subjects readily provide saliva, if asked, and obtaining saliva or another oropharyngeal biological sample such as sputum requires no additional skills. In fact, as any of a number of commercial genotyping services have demonstrated, saliva DNA can be obtained by individuals at home and returned via mail, eliminating even the need for any in person contact.

For example, the methylation set-points (i.e. in the absence of outside influences) differ between the two tissues present in saliva, with the Illumina array data, simply for example, indicating that the set points for blood and buccal DNA differ by approximately 5-6%. As a result, for example, in the absence of compensating for cellular heterogeneity, non-smoking subjects with a high proportion of buccal cell content in their saliva DNA may appear as non-smokers, while lightly smoking subjects who have an unexpectedly high proportion of blood DNA in their saliva could appear as non-smokers. Thus, a number of loci are described herein that can be used to make such a correction.

To compensate for the differential set points of methylation in different tissues, researchers have used complicated, multi-locus methods to correct for similar heterogeneity (e.g., Houseman et al., 2012, BMC Bioinform., 13:86). Whereas these methods can be applied to genome wide assessments of methylation, they cannot be applied to less than genome wide assessments of methylation. Therefore, developing a method that can assess cellular heterogeneity using data from one or a few loci could eliminate the need for this expensive and complex method and also allow more rapid methylation sensitive quantitative or digital polymerase chain reaction (PCR) assessments to measure DNA methylation in saliva, allowing a greater breadth of subjects to be sampled much more easily and affordably.

This disclosure describes an assay that accurately measures cellular heterogeneity and allows the use of saliva DNA methylation assessments to perform equivalently to those conducted on whole blood. It is novel because it utilizes a locus in which there is relatively little difference in methylation between different types of white blood cells and at which there is a large difference in methylation between white blood cells and buccal cells. By measuring methylation at one or more of the DMR loci described herein and using an algebraic equation, the relative contribution of buccal and white blood cell DNA to saliva DNA can be directly assessed and used to correct other methylation assessments of health condition-related loci to impute health status more accurately.

It would be understood by a skilled artisan that a DMR loci having an absolute differential methylation amount between white blood cells and buccal cells of at least 0.5 can be used as described herein, but it also would be understood by a skilled artisan that those DMR loci having an absolute differential methylation amount between white blood cells and buccal cells of at least 0.7, 0.8, 0.9 or higher will significantly improve the accuracy of the final determination.

A number of DMR loci meeting this criteria were identified, and are shown in Table 2. These include DMR11, identified by cg25574765 (sometimes referred to as “DMR11(cg25574765)”); DMR20, identified by cg03841065 (sometimes referred to as “DMR20(cg03841065)”); DMR11, identified by cg10511890 (sometimes referred to as “DMR11(cg10511890)”); DMR12, identified by cg08075204 (sometimes referred to as “DMR12(cg08075204)”); DMR7, identified by cg24620436 (sometimes referred to as “DMR7(cg24620436)”); DMR20, identified by cg07598052 (sometimes referred to as “DMR20(cg07598052)”); DMR16, identified by cg04921315 (sometimes referred to as “DMR16(cg04921315)”); DMR11, identified by cg26427109 (sometimes referred to as “DMR11(cg26427109)”); DMR2, identified by cg00438740 (sometimes referred to as “DMR2(cg00438740)”); DMR6, identified by cg09344348 (sometimes referred to as “DMR6(cg09344348)”); DMR11, identified by cg08141395 (sometimes referred to as “DMR11(cg08141395)”); DMR10, identified by cg24681845 (sometimes referred to as “DMR10(cg24681845)”); DMR19, identified by cg22824635 (sometimes referred to as “DMR19(cg22824635)”); DMR4, identified by cg14516100 (sometimes referred to as “DMR4(cg14516100)”); and DMR1, identified by cg20820767 (sometimes referred to as “DMR1(cg20820767)”). Two DMR loci, the DMR11 loci identified by cg08141395, referred to herein as “DMR11,” and the DMR16 loci identified as the CpG immediately next to cg02614661, referred to herein as “DMR16,” were selected to demonstrate the effectiveness of the ability to correct for cellular heterogeneity described herein.

The relative contribution of white blood cells (X) to the total DNA sample in saliva was determined for a CpG dinucleotide referred to as DMR11 (targeted herein using the probes shown in SEQ ID NO:16 or 17) by solving the following equation:

DMR11(obs)=(0.01X+0.99(1−X))

where DMR11(obs) is the observed methylation signal of DMR11 in the heterogeneous saliva sample, and 0.01 and 0.99 are the fractional methylation values of DMR11 in white blood cells and buccal cells, respectively.

Similarly, the relative contribution of white blood cells (X) to the total DNA sample in saliva was determined for a CpG dinucleotide referred to as DMR16 (targeted herein using the probes shown in SEQ ID NO:8 or 9) by solving the following equation:

DMR16(obs)=(0.97X+0.18(1−X))

where DMR16(obs) is the observed methylation signal of DMR16 in the heterogeneous saliva sample, and 0.97 and 0.18 are the fractional methylation values of DMR16 in white blood cells and buccal cells, respectively.

Compensation for this source of noise through the use of one or more DMR markers has been shown to improve the prediction of both smoking and drinking, which supports the use of this marker for saliva DNA analyses. This disclosure demonstrates that methylation assessment of any of several differentially methylated regions (DRMs) allows nearly perfect correction for cellular heterogeneity in saliva.

The target sequence for which the methylation status is determined and used to correct for cellular admixture can be any one or more of the thousands of CpG dinucleotides present in the genome. As described in Lowe et al. (2013, Epigenetics, 8(4):445-54), there are 33,998 differentially methylated regions in autosomal DNA, with 29,418 being hypomethylated in buccal cell but only 4,580 being hypomethylated in blood DNA.

The CpG residue for whom the correction approach can be applied to better understand the methylation in either the blood or buccal cell contribution to saliva DNA is any sequence whose methylation set point differs by more than 1% between blood and buccal DNA. For example, the target sequence can be one or more of the CpG dinucleotides found within the aryl hydrocarbon receptor repressor (AHHR) gene and can be indicative of whether or not an individual uses nicotine (see, e.g., U.S. Pat. No. 9,273,358); the target sequence can be within the promoter sequence of the EDARADD, TOMILI, or NPTX2 genes and can be indicative of the age of an individual (see, e.g., U.S. Pat. No. 10,435,743); or the target sequence can be CNKSR1 and can be indicative of heart or cardiovascular disease (see, e.g., WO 2017/214397). Typically, the methylation status of the target sequence is indicative of some aspect of health, environmental exposure, and/or diagnostic status.

Methods of determining the methylation status of a target nucleic acid sequence and/or a DMR loci (e.g., one or more CpG dinucleotides or of a CpG island within a target sequence or a DMR loci) are known in the art. It would be appreciated that the most common method for evaluating the methylation status of DNA begins with a bisulfite-based reaction on the DNA (see, for example, Frommer et al., 1992, PNAS USA, 89(5):1827-31). Commercial kits are available for bisulfite-modifying DNA. See, for example, EpiTect Bisulfite or EpiTect Plus Bisulfite Kits (Qiagen).

Following bisulfite modification, the nucleic acid can be amplified. Since treating DNA with bisulfite deaminates unmethylated cytosine nucleotides to uracil, and since uracil pairs with adenosine, thymidines are incorporated into DNA strands in positions of unmethylated cytosine nucleotides during subsequent PCR amplifications.

In some embodiments, the methylation status of a nucleic acid sequence can be determined using one or more nucleic acid-based methods. For example, an amplification product of bisulfite-treated DNA can be cloned and directly sequenced using recombinant molecular biology techniques routine in the art. Software programs are available to assist in determining the original sequence, which includes the methylation status of one or more nucleotides, of a bisulfite-treated DNA (e.g., CpG Viewer (Carr et al., 2007, Nucl. Acids Res., 35:e79)). Alternatively, amplification products of bisulfite-treated DNA can be hybridized with one or more oligonucleotides that, for example, are specific for the methylated, bisulfite-treated DNA sequence, or specific for the unmethylated, bisulfite-treated DNA sequence. In some instances, a methylation-specific PCR assay can be used to determine the methylation status of a target sequence and/or a DMR loci.

In some embodiments, the methylation status of DNA can be determined using a non-nucleic acid-based method. A representative non-nucleic acid-based method relies upon sequence-specific cleavage of bisulfite-treated DNA followed by mass spectrometry (e.g., MALDI-TOF MS) to determine the methylation ratio (methyl CpG/total CpG) (see, for example, Ehrich et al., 2005, PNAS USA, 102:15785-90). Such a method is commercially available (e.g., MassARRAY Quantitative Methylation Analysis (Sequenom, San Diego, Calif.)).

Any of the DMR loci identified herein (e.g., DMR11, DMR16) or a different DMR locus identified using the methods described herein can be included in an article of manufacture. For example, an article of manufacture can include a first pair of DMR primers, and at least one DMR probe that detects either a methylated or an unmethylated CpG dinucleotide. In some instances, an article of manufacture can include a first pair of DMR11 primers, and at least one DMR11 probe that detects either a methylated or an unmethylated CpG dinucleotide. In some instances, an article of manufacture can include a first pair of DMR16 primers, and at least one DMR16 probe that detects either a methylated or an unmethylated CpG dinucleotide.

It would be understood that any number of additional components can be included in an article of manufacture. For example, an article of manufacture can include at least one additional probe that detects either the methylated or the unmethylated CpG dinucleotide (i.e., the opposite of the at least one probe contained in the article of manufacture). It would be appreciated that a second pair of primers can be used in an amplification reaction and can be included in an article of manufacture as described herein. In addition, an article of manufacture can include, without limitation, reagents for bisulfite converting nucleic acid, reagents for amplifying nucleic acid, and/or reagents for sequencing nucleic acid.

Representative combinations of primers and probes are described herein. For example, an article of manufacture can include the pair of DMR11 primers shown in SEQ ID NOs:12 and 15 and at least one DMR11 probe shown in SEQ ID NO:16 or 17. Such an article of manufacture also can include the pair of DMR11 primers shown in SEQ ID NOs:13 and 14. Alternatively, an article of manufacture can include the pair of DMR16 primers shown in SEQ ID NOs:3 and 5 and at least one DMR16 probe shown in SEQ ID NO:7 or 8. Such an article of manufacture also can include the pair of DMR16 primers shown in SEQ ID NOs:4 and 6.

Methods are described herein that can be used to identify suitable DMR sequences and develop the associated formula in essentially any heterogeneous biological sample that contains blood as one of the major components. While such methods are illustrated herein using saliva DNA, which contains blood and buccal cell DNA, such methods can be applied to virtually any type of biological sample from the oropharyngeal fossa but also can be applied to biological samples such as urine.

The first step of the method is to compare the methylation status of a large number (i.e., a plurality) of loci in a first cellular or tissue component of the heterogeneous biological sample and the methylation status of a large number (i.e., a plurality) of loci in a second cellular or tissue component of the heterogeneous biological sample. The second step of the method is to identify one or more loci that are differentially methylated within the plurality of loci in the first cellular or tissue component of the heterogeneous biological sample relative to the plurality of loci in the second cellular or tissue component of the heterogeneous biological sample.

As described herein, the identified loci should have an absolute difference of at least 0.5 (e.g., at least 0.6, at least 0.7, at least 0.8, at least 0.9, at least 0.95, or at least 0.99) between the methylation status of the loci in the first cellular or tissue component and the methylation status of the loci in the second cellular or tissue component. As described herein, this method identifies one or more DMR loci and the associated formula that can be used to correct for the cellular heterogeneity that is found in that particular heterogeneous biological sample.

In accordance with the present invention, there may be employed conventional molecular biology, microbiology, biochemical, and recombinant DNA techniques within the skill of the art. Such techniques are explained fully in the literature. The invention will be further described in the following examples, which do not limit the scope of the methods and compositions of matter described in the claims.

### EXAMPLES

### Example 1—Chromosome 16 DMR Region Assay

### DMR6 Pre-Amp

The following PCR conditions were used: 10× buffer, dNTPs, and 95° C.×4 min, then 20 cycles of 94° C. for 30 sec, 60° C. for 30 sec and 72° C. for 30 sec. DMR F1 and DMR R1 primers were used at a net concentration in the PCR reaction of 0.1 μM, and 3 μl of bisulfite converted DNA was used as the template. A total volume of 10 μl was used.

| CHRM 16 DMR Pre Amp | | 20X | 60 X | 110 X | 220 x |
|---|---|---|---|---|---|
| | 10X | 20 | 60 | 110 | 220 |
| | dNTP | 30 | 90 | 165 | 330 |
| 20 μM Primer | DMR F1 | 1 | 3 | 5.5 | 11 |
| | DMR R2 | 1 | 3 | 5.5 | 11 |
| | Water | 90 | 270 | 495 | 990 |
| | Taq | 2 | 3.5 | 6 | 12 |
| template DNA | 3 μl each well | | | | |

After PCR, each reaction was diluted to 50 μl with water, and 5 μl of the resulting solution to use as the template for RT-PCR.

### RT-PCR Conditions:

The following RT-PCR conditions on a 9700 (or equivalent) were used with Universal PCR Master mix: 95° C.×10 min, then 40 cycles of 95° C. for 15 sec, 55° C. for 15 sec and 60° C. for 1 min. A total volume of 10 μl was used.

| | | |
|---|---|---|
| Primer Conc: | DMR F2 | 300 nM |
| | DMR R3 | 300 nM |
| | Probes | 250 nM each |

| | | | For 5 μl of liquid Target | | | |
|---|---|---|---|---|---|---|
| | | | and 5 μl of 2x Mix | | | |
| RT-PCR Mix | | | 20X | 60 X | 110 X | 220 X |
| Master Mix | | | 90 | 270 | 495 | 990 |
| 20 μM Primer | DMR F2 | | 3 | 9 | 16.5 | 33 |
| | DMR R3 | | 3 | 9 | 16.5 | 33 |
| 10 μM probe | 56-Fam | Meth | 5 | 15 | 27.5 | 55 |
| | 56-JOEN | Unmeth | 5 | 15 | 27.5 | 55 |

| Sequences | | |
|---|---|---|
| DMR16 (non-bisulfite converted); corresponds to chr16:87877794- | | |
| 87878853 of the hg19 assembly | | |
| (SEQ ID NO: 1) | | |
| TTGTCCCTAAGAGGCATCTTCCTCAGGGGCTGGTGGAGCTGCCATGAAAGCAAACGCACAGC | | |
| CAAACCCCGGGTGGGGGGAAGGCAAACTGCAAACGCCGCGGCGACCCCGGCACAGCAGCCCT | | |
| GTCAGCAGGATTCCCCCGAGAGCGGGGTAATTGCGGTGGGAACGAGCGCTCCAAAGGCCCTG | | |
| GGGAGATGATTTCAGGGAAAAGTGGCCTTGATCCCTGAGTCAGGCAGATGCGGCCATGGGAA | | |
| CCATCCACCCCGAGGCTGGAGGGGAGACTCCGCCGGTGGCTAAAGCCATCCTGCTGACGGGG | | |
| CCCAGGGACGCCCCCAGTGGCCAAACGCACGTGGGAACGGGATCTTCCCCCTCCTCTGTGAT | | |
| GCGGCCAACCCTCCAAGCCTCTGGCTCCTGACTCAGAGGACGATGTCTCCCCATGAACGCAG | | |
| TGTCCCTGGAGAGAAGAGCCTGCCCAGCGTGGGGAACATGGGAATGTGGAGATGGAGGGCAT | | |
| 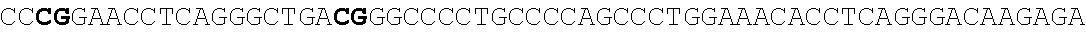 | | |
| GTCACTCCTCAAGCGCGGACTTTCCACTGTGCTGGGGCCTTCCGCCTTCCAACCACTCTGGC | | |
| CCCTTGGGGCTCTAGGTGGAGTGTGCTGAACAGTGTCCCCAAAATTCACGTCCAGTGGAGCT | | |
| GCGGGATGTGTCCTAGCTGCAAATGCGGTCTCCGCAGGTGCAATTAGCGAAGGACCTTGAGA | | |
| TGAGATCATCCCGGATTAGAGTAGGCACTAAGGCCGACGACAAGTGTCCTCAGCGACACAGA | | |
| GACAGATCCAGGGGAGACAGAGCCAGAGCCAGCCCAGGATGCCTGGAGCCACGGGCAGCTGG | | |
| AAGGGGAAGGTGGCACCTTGGGTCTGGACCTCTGGCTGCCAGGACCAGGACCGTCTGCATGT | | |
| CTGTTGCTAAAGCCAGTCTGTAGGCATCTGTCACCACACATGAGGGGCCAACCGTGCCACCC | | |
| AGGGGAGACCTCCCGGCTTCTCAGTGCATCAGGCATGGTCAGGGGCAGGACAGCTGCAGCTC | | |
| ACACCC | | |
| DMR16 (bisulfite converted; assumes complete methylation of the | | |
| CG sites. In other words, all “C” nucleotides not immediately 5′ | | |
| of a “G” nucleotide (i.e., CpG) were converted to “T” nucleotides | | |
| due to bisulfite treatment). | | |
| (SEQ ID NO: 2) | | |
| TTGTTTTTAAGAGGTATTTTTTTTAGGGGTTGGTGGAGTTGTTATGAAAGTAAACGTATAGT | | |
| TAAATTTCGGGTGGGGGGAAGGTAAATTGTAAACGTCGCGGCGATTTCGGTATAGTAGTTTT | | |
| GTTAGTAGGATTTTTTCGAGAGCGGGGTAATTGCGGTGGGAACGAGCGTTTTAAAGGTTTTG | | |
| GGGAGATGATTTTAGGGAAAAGTGGTTTTGATTTTTGAGTTAGGTAGATGCGGTTATGGGAA | | |
| TTATTTATTTCGAGGTTGGAGGGGAGATTTCGTCGGTGGTTAAAGTTATTTTGTTGACGGGG | | |
| TTTAGGGACGTTTTTAGTGGTTAAACGTACGTGGGAACGGGATTTTTTTTTTTTTTTGTGAT | | |
| GCGGTTAATTTTTTAAGTTTTTGGTTTTTGATTTAGAGGACGATGTTTTTTTATGAACGTAG | | |
| TGTTTTTGGAGAGAAGAGTTTGTTTAGCGTGGGGAATATGGGAATGTGGAGATGGAGGGTAT | | |
| 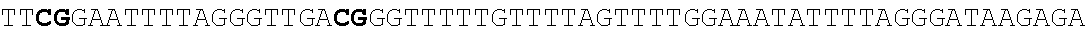 | | |
| GTTATTTTTTAAGCGCGGATTTTTTATTGTGTTGGGGTTTTTCGTTTTTTAATTATTTTGGT | | |
| TTTTTGGGGTTTTAGGTGGAGTGTGTTGAATAGTGTTTTTAAAATTTACGTTTAGTGGAGTT | | |
| GCGGGATGTGTTTTAGTTGTAAATGCGGTTTTCGTAGGTGTAATTAGCGAAGGATTTTGAGA | | |
| TGAGATTATTTCGGATTAGAGTAGGTATTAAGGTCGACGATAAGTGTTTTTAGCGATATAGA | | |
| GATAGATTTAGGGGAGATAGAGTTAGAGTTAGTTTAGGATGTTTGGAGTTACGGGTAGTTGG | | |
| AAGGGGAAGGTGGTATTTTGGGTTTGGATTTTTGGTTGTTAGGATTAGGATCGTTTGTATGT | | |
| TTGTTGTTAAAGTTAGTTTGTAGGTATTTGTTATTATATATGAGGGGTTAATCGTGTTATTT | | |
| AGGGGAGATTTTTCGGTTTTTTAGTGTATTAGGTATGGTTAGGGGTAGGATAGTTGTAGTTT | | |
| ATATTT | | |
| DMR F1 | GTAGTGTTTTTGGAGAGAAGAG 59 | (SEQ ID NO: 3) |
| DMR F2 | TATGGGAATGTGGAGATGG 59 | (SEQ ID NO: 4) |
| DMR R1 | CACACTCCACCTAAAACCC 61 | (SEQ ID NO: 5) |
| DMR R3 | CTCTCTTATCCCTAAAATATTTCCA 58 | (SEQ ID NO: 6) |
| | 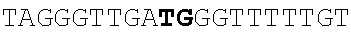 | (SEQ ID NO: 7) |
| | 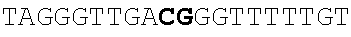 | (SEQ ID NO: 8) |

C allele probe (meth): /56FAM/TT+G+A+C+GG+G+TTT/IBkFQ/ (65/49) delta Tm=16° C. (SEQ ID NO:9)

T allele (unmeth): /5JOE/TT+GA+T+G+G+GTTT (63.45/51.83) delta Tm=11.62° C. (SEQ ID NO:10)

### Example 2—Application of the DMR16 Assay to Adjust for Methylation within AHRR Using Both Saliva and Blood DNA is a Powerful Predictor of Smoking Status

### Results

The demographic and clinical characteristics of the 418 subjects who participated in the study are given in Table 1. The control group, all of whom denied any form of substance consumption over the past year, was largely White, middle-aged and mostly female (56%). In contrast, the smoking subjects, while largely the same age, were disproportionately male (64%) and much more ethnically diverse with 150% of subjects reporting African-American ancestry. Finally, like the control subjects, the 31 subjects who had reported consuming at least 100 cigarettes in their lifetime, but not smoking in the past 10 years, were largely female (58%). However, they were exclusively White and significantly older than the other two cohorts (p<0.0001).

TABLE 1
Clinical and Demographic Characteristic of the Subjects

| | Controls | Smokers | Quitters |
|---|---|---|---|
| N | 154 | 233 | 31 |
| Age | 41.3 ± 14.7 | 41.2 ± 11.7 | 56.5 ± 12.2 |
| Ethnicity | | | |
| White | 139 | 188 | 31 |
| African American | 3 | 34 | 0 |
| Other | 12 | 11 | 0 |
| Gender | | | |
| Male | 68 | 150 | 13 |
| Female | 86 | 83 | 18 |
| Cigarettes per day | | | |
| Past Month | — | 16.3 ± 10.6 | — |
| Past 6 Months | — | 17.2 ± 11.3 | — |
| Past Year | — | 17.5 ± 11.4 | — |
| Life Consumption Pack Yr | | 15.3 ± 14.6 | |
| Average WB cg05575921 | 86.2% ± 2.9 | 49.2% ± 16.4 | 82.0% ± 6.6 |
| Average Saliva cg05575921 (%) | 73.5% ± 6.5 | 38.3% ± 15.4 | 69.2% ± 7.3 |
| Some Cannabis Past Year? | 0 | 91 | 0 |

As a first step, the relationship between cg05575921 methylation in whole blood was analyzed with respect to key demographic variables of the subjects. In the control subjects, there was no significant relationship between cg05575921 methylation with age, but there was a significant relationship between methylation status and gender, with females tending to have a slightly higher methylation (86.6%±2.3 vs 85.6%±3.4, p<0.05). In contrast, there was no relationship between gender and methylation in the smoking subjects, but there was a significant negative relationship between age and cg05575921 methylation (p<0.007). Finally, in the smoking subjects, the mean methylation of the African-American subjects did not differ from that of the White subjects (50.1%±14.2 vs 48.8%±16.8).

The relationship of cg05575921 methylation in whole blood to group status was examined. The average DNA methylation of the non-smoking subjects and those who had quit for at least 10 years did not significantly differ. However, the average value of both the Control and Quitter cohorts were significantly greater than those of the Smokers.

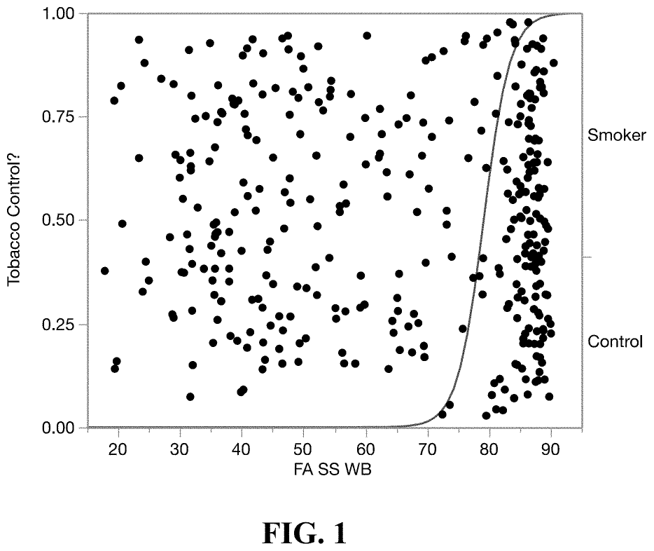

As the final step of the initial analyses of just the whole blood methylation data, the capacity of cg05575921 methylation to predict current smoking status and average daily cigarette consumption was examined. FIG. 1 is a logistic plot of the distribution of cg05575921 methylation as a function of Smoker or Control status. As the figure shows, all but two of the controls have methylation greater than 78% while only 12 of the Smokers have values of >78%. Using a standard Receiver Operating Characteristic (ROC) approach to analyze these data, the area under the curve (AUC) for predicting smoking status was 0.99.

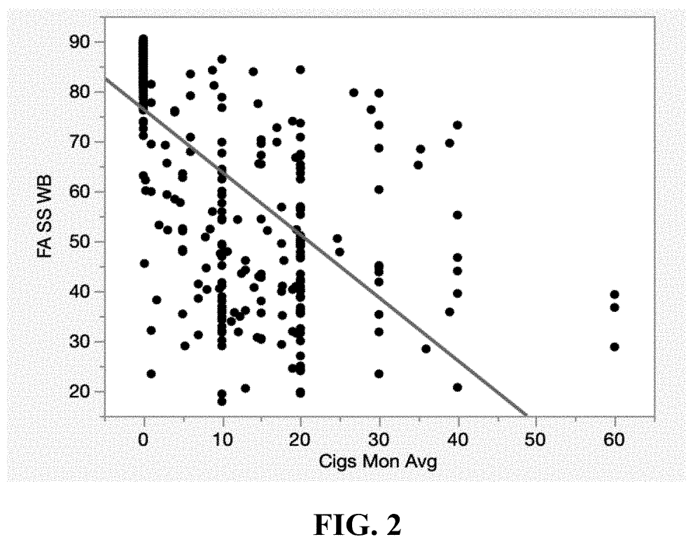

The relationship between average daily cigarette consumption over the past month and cg05575921 methylation is shown in FIG. 2. Consistent with prior reports, increasing cigarette consumption was significantly negatively correlated with cg05575921 methylation (n=355, Adjusted R²=0.405, p<0.0001) with a linear fit to the model showing that every one percent decrease of methylation being associated with a 1.2 increase in the number of cigarettes consumed per day. Similar results were seen with respect to regression analyses of cigarette consumption when averaged over the past six months and one year time windows. Finally, the relationship between total pack year consumption and DNA methylation at cg05575921 methylation in whole blood was analyzed. Once again, using simple linear model that uses only cg05575921 methylation to predict pack year consumption, the relationship was highly significant (n=355, Adjusted R²=0.405, p<0.0001) with each one percent decrease of cg05575921 methylation below 76% being associated with a 0.96 increase in lifetime pack year smoking consumption. The addition of age to model further improved the variance predicted by the model to 0.43.

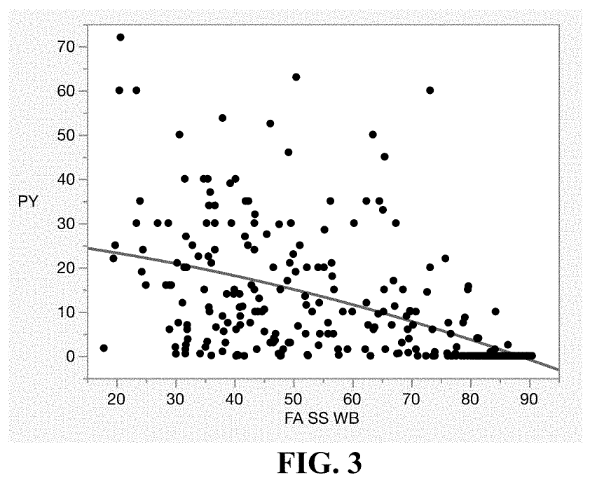

Finally, we analyzed the relationship of methylation to consumption (pack years, see FIG. 3). Once again, a curvilinear model produced a better fit to the model than a simple linear fit with the amount of demethylation observed steadily decreasing with each increasing pack year of consumption.

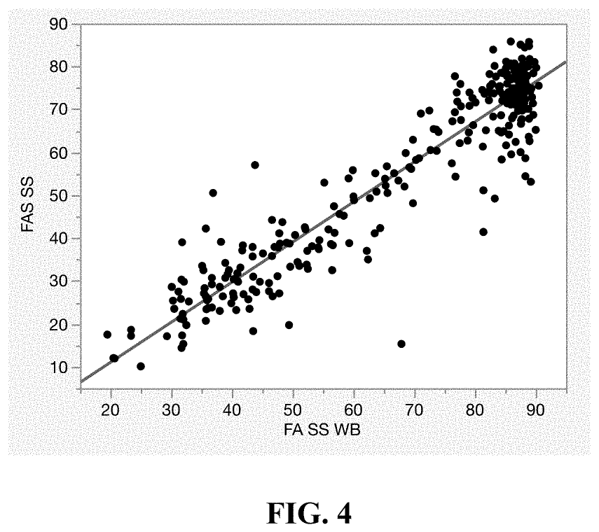

The second set of analyses focused on the relationship of cg05575921 methylation in saliva to group status. As a first step, the relationship of cg05575921 levels was analyzed in whole blood and compared to those of saliva for 274 subjects for whom we have methylation data in both whole blood and saliva DNA. FIG. 4 illustrates the results of that relationship. Overall, cg005575921 values in the two preparations were highly correlated with a linear fit of the data producing an adjusted R² of 0.89 (n=274, p<0.0001).

Although the above linear fit model of cg05575921 methylation is quite strong, considerable variance in the relationship between these two measurements remains unexplained. One possible contributor to the imperfect correlation of cg005575921 levels in whole blood as compared to those of saliva may result from the heterogeneity of cell types in saliva. Saliva DNA contains a variable proportion of bacterial and human DNA. The human portion of that DNA is derived from two principal cell types. The majority is from white blood cells that marginate into saliva via the gums or the salivary glands. The remainder of the DNA is contributed by sloughed buccal cells. If the tissue specific set points of the buccal and whole blood DNA significantly differ, it is conceivable that part of the reason of the imperfect relationship is differing ratios of blood vs buccal cells in the saliva DNA preparations.

To help address this problem, we have developed a proprietary marker, termed DMR16, that assess DNA methylation at a locus that is 18% methylated in buccal cells (n=3 data not shown) and 97% in whole blood DNA (n=270, data not shown) with no evidence of genetic variation that affects methylation set point. To better understand and visualize this source of noise, using the DMR16 data, the percentage of whole blood contribution to the human DNA in the saliva sample was first calculated, then tested whether the addition of DMR16 information to the cg05575921 methylation data would improve the ability of the model to predict smoking status.

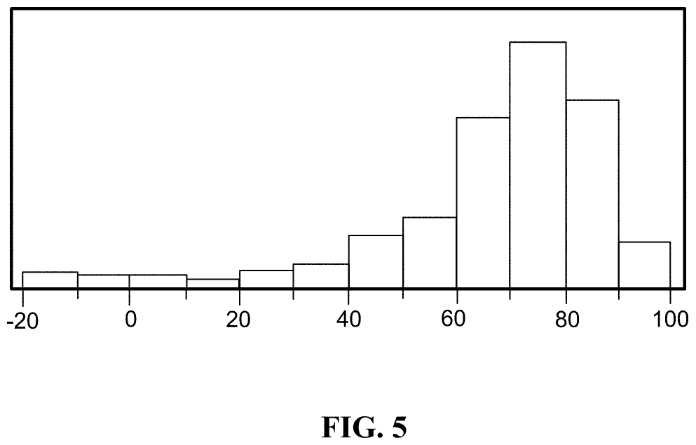

FIG. 5 illustrates the imputed percentage of white blood cell DNA in each saliva sample (n=301). Using this approach, the average contribution of white blood cell DNA to the total human contribution is 67%±21.

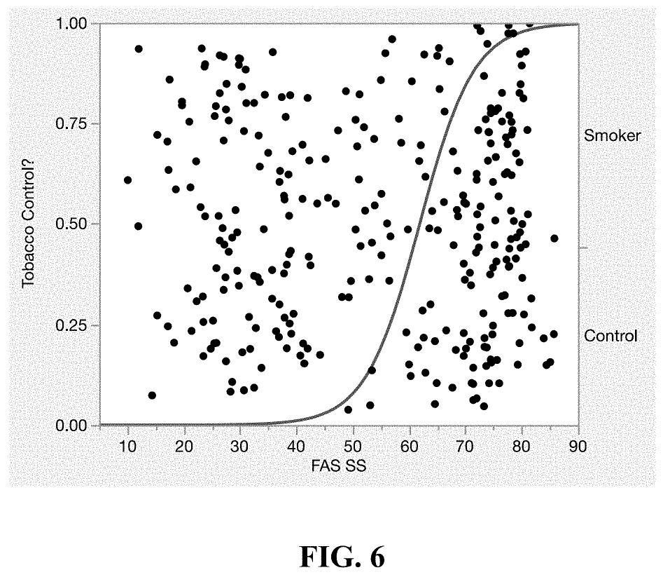

The predictive power of only saliva DNA methylation at cg05575921 alone was tested and in combination with DMR16 information to predict smoking status. FIG. 6 illustrates the relationship of saliva DNA methylation to class status. As illustrated by the logistic plot, the spread of cg05575921 values in saliva DNA for the controls is considerably greater than that for the whole blood values. Using DNA from whole blood, the Receiver Operating Characteristic (ROC) area under the curve (AUC) for predicting smoking status was 0.99 with the correlation between cg05575921 methylation and cigarettes per day being −0.64. Using DNA from saliva, the unadjusted ROC AUC for predicting smoking was 0.965 with the correlation between cg05575921 methylation and cigarettes per day consumption being −0.61. The addition of DMR16 information to the model improves the predictive power even further with an AUC for saliva DNA of 0.985.

### Methods and Materials

The clinical data and biomaterials used in this study were collected using two separate, National Institutes of Health funded, protocols that were approved by the Western Institutional Review Board (WIRB®; WIRB Protocols #20162083 and WIRB #20160135).

The clinical data and biomaterials from three distinct groups of actively smoking subjects were used in this study. The first set of active smokers was recruited from a previously described study of alcohol consumption that recruited subjects from one of three Iowa substance use treatment organizations Center for Alcohol and Drug Services (CADS, Davenport, Iowa), Prelude Behavioral Services (campuses in Iowa City and Des Moines, Iowa) and Alcohol and Drug Dependency Services of Southeast Iowa (ADDS, Burlington, Iowa). The second set of active smokers was recruited from a study of smoking cessation conducted at only the CADS (Davenport, Iowa) site. After consent, each subject was interviewed with an abbreviated form of the commonly used Semi Structured Assessment for the Genetics of Alcoholism (SSAGA) and our Substance Use Questionnaire (Philibert et al., 2014, Epigenetics, 9:1-7), which is a focused inventory of substance use consumption over the past year. Then after interview, each of the subjects was phlebotomized in order to provide biomaterials for the current study. In every case, the self-report smoking was confirmed by serum cotinine determinations as described below.

The clinical data and biomaterials from the non-smoking “Controls” and those subjects (“Quitters) who report quitting smoking more than 10 years previously were obtained from the control arm of the alcohol consumption study. These control subjects were solicited via an e-mail recruitment sent to the University of Iowa staff and student community that stipulated participation in this portion of the study was dependent on abstinence from alcohol or any non-nicotine form of substance abuse in the prior year. In total, 163 subjects of the 212 subjects consented in this control arm of the study denied lifetime consumption of more than 100 cigarettes or other forms of smoking, while 31 other subjects reported at consuming at least 100 cigarettes in their lifetime, but denied any form of smoking in the past 10 years. Each of these subjects were consented, then interviewed with the SSAGA and the Substance Use Questionnaire. After the interview, each of the subjects was then phlebotomized to provide biomaterials for this study. An additional group of 18 subjects reported some form of cannabis or tobacco consumption in this protocol in the past 10 years (total n=212). However, since their form of substance use precluded easy categorization and generally did not involve cigarettes, their data was excluded from the study.

Serum cotinine and cannabinoid levels were determined for all subjects using enzyme linked immunoassay kits from AbNova (Taiwan) according to manufacturer's direction. As part of the process of screening the 212 subjects enrolled in the control arm of the alcohol consumption study, cotinine values of >2 ng/ml were found for serum samples from nine participants (6 males, 3 females) who denied any use of any nicotine containing product in the past year. Their data was excluded from this study. As a result, the total number of non-smoking controls was reduced from 163 to 154 subjects.

Data analysis: Methylation status at cg05575921 and DMR16 were determined as previously described (Philibert et al., 2018, Frontiers of Genetics and Epigenetics, 9:137). In brief, 1 μg of DNA of either whole blood or saliva DNA was bisulfite converted using a EpiTect® Fast DNA kit from Qiagen (Germany) according to manufacturer's directions. An aliquot of each of these modified DNA samples was pre-amped, diluted 1:3000 with molecular grade water, and partitioned into ˜1.5 nanoliter aqueous droplets encased in oil using an automated droplet generator. DNA amplicons contained within these droplets were then PCR amplified using proprietary primer probe sets (Smoke Signature® or DMR16) for each locus from Behavioral Diagnostics (Coralville, Iowa) and universal digital PCR reagents from Bio-Rad (Carlsbad, Calif.). The number of droplets containing amplicons with at least one “C” allele (representing an originally methylated CpG residue), one “T” allele (which represents a CpG residue that was unmethylated) or neither allele was then determined using a Bio-Rad QX-200 droplet reader. Percent methylation was calculated using Quantisoft software by fitting the observed ratios to a Poisson distribution. Relative contribution of white blood cell contribution (X) to the total DNA sample was determined by solving the equation of DMR16(obs)=(0.97X+0.18(1−X)) where DMR16(obs) is the observed methylation signal in the saliva sample, and 0.97 and 0.18 are the fractional methylation values of DMR16 in white blood cells and buccal cells, respectively.

Standard linear regression was used to examine the relationship of methylation status to age and gender. Boxplots were constructed to display the distribution of methylation status by gender. The primary analyses were conducted using logistic regression where the outcome was smoking status and each model was adjusted for age and gender.

To demonstrate the predictive capability of smoking status using the ddPCR assay, data from all 177 subjects (98 smokers and 78 controls) were randomly split into training (70%) and testing datasets (30%). The training and testing datasets consisted of 125 (70 smokers and 55 non-smokers) and 52 subjects (29 smokers and 23 non-smokers), respectively. A binary logistic regression model was fitted in R using training set data to predict the probability of being a smoker using DNA methylation at cg05575921. By assigning a false negative misclassification cost twice as much as a false positive misclassification cost, the prediction probability cutoff was determined to be 0.1467216. The trained model was then saved for testing on the test set. This approach was repeated to include age and gender in the prediction model. The probability cutoff when age and gender were included was 0.3821462.

Other quantitative non-genome wide analyses of both array and ddPCR derived methylation data were conducted using JMP Version 10 software (SAS, Cary, N.C. USA).

### Example 3—Application of DMR16 Correction for Evaluating Alcohol Use from a Saliva Sample

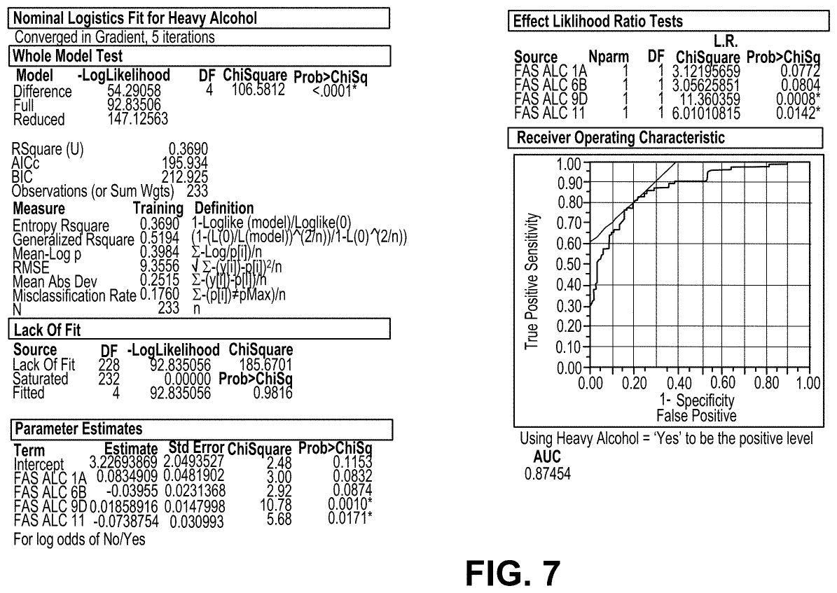

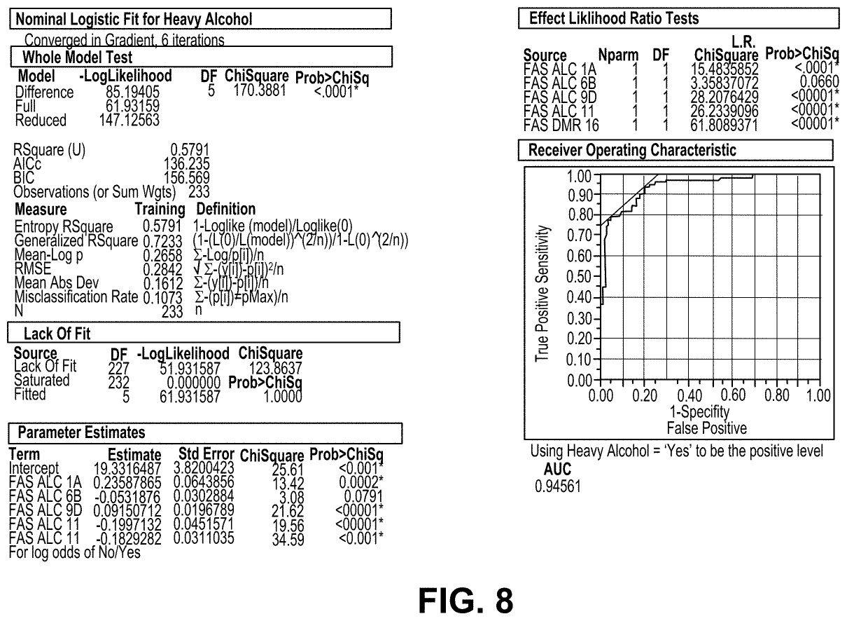

A similar approach was applied to data from subjects who use or don't use alcohol. See, for example, Philibert et al., 2019, J. Ins. Med., 48:1-13. The data from these experiments in saliva is shown in the absence of DMR16 correction (FIG. 7) and in the presence of DMR16 correction (FIG. 8). As demonstrated, the ROC increases significantly from 0.87 in the absence of DMR16 correction to 0.95 in the presence of DMR16 correction.

### Example 4—Genome Wide Studies Show the Existence of Numerous Loci Capable of Correcting for Cellular Heterogeneity

To demonstrate the point that there are many loci similar to DMR16 that can be used to correct for heterogeneity using simple methylation-sensitive digital PCR or sequencing techniques, two sets of genome wide data were obtained and analyzed. First, the genome wide data generated using the Illumina human methylation 450 k bead chip array (aka “450K array”) from cells and buccal scrapings (Lowe et al., 2013, Epigenetics, 8:445-54) were analyzed to determine the number of differentially methylated sites. Altogether, data from 441,946 CpG probes from the 450K array were available for analysis. Second, using the Infinium Methylation EPIC Array (aka “Epic array”), methylation was determined in 15 paired samples of whole blood and saliva from 15 subjects who participated in studies of substance use (Philibert et al., 2018, Am. J. Med. Genet. Part B: Neuropsych. Genet., 177:479-88). The preparation of that paired whole blood/saliva methylation data followed the standard protocols as described in Philibert et al. (2018, supra). After processing, data from 848,525 CpG probes from the Epic array were available for analysis.

At a macroscopic level, the genome wide correlation of methylation within the group of 15 blood samples was 0.987, while the correlation among the saliva samples, which include various mixtures of buccal and whole blood cells, was only 0.977. Finally, as expected, the genome wide correlation between the paired samples was also very high, at 0.988.

At the individual locus level, however, the average correlation between methylation of whole blood and saliva methylation is much lower, at 0.24. Although this discrepancy may be confusing at first glance, genome wide methylation measures are typically highly correlated because the strength of the correlation is driven by differences between extremely hypermethylated and unmethylated regions. That is why the correlation between the methylation values for the whole blood samples from 15 unrelated individuals discussed above is so high (i.e. 0.987).

At the individual locus level, however, the contrast is more discrete, and instead, variation affecting the correlation between methylation from paired whole blood and saliva samples arises from at least two key sources: a) measurement error and b) differences attributable to cellular heterogeneity. The former can be substantial, with some authors citing error effects reaching 6%. In contrast, the amount of difference contributed by cellular heterogeneity in saliva samples is locus dependent and highly influenced by the methylation set point of the two tissues that contribute DNA to saliva, namely blood cells and buccal cells. Lowe and colleagues demonstrated profound effects of cellular origin on methylation set point (Lowe et al., 2013, Epigenetics, 8:445-54), however, a limitation was that they used purified subcellular components of whole blood (CD14, CD4 and CD34).

To circumvent the purified cell-based approach and get an idea of the number of markedly differentially methylated sites between whole blood DNA and buccal DNA, the whole blood data from our Epic assessment was combined with the prior buccal data from Lowe et al. (2013, supra). Not all probes in the 450K array are present in the Epic array; 399,470 probes overlapped between the two arrays in the data set used herein. The average DNA methylation at these overlapping 399,470 loci in blood cells was 44.5%, while the average DNA methylation at these same loci in buccal cells was 49.4%. Therefore, the average absolute difference in methylation status between whole blood and saliva samples across these nearly 400,000 sites was 13%. In total, methylation differed by 70% or more at 3,807 CpG loci, with methylation at cg02614661, the site immediately next to the CpG site used in the DMR16 assay, being only the 4744^{th} highest ranked site.

Table 2 lists the 15 most significantly differentially methylated sites from this comparison. Please note that the absolute difference of methylation at each of these sites is substantially higher than the absolute difference between buccal DNA and whole blood DNA methylation at the DMR16 locus (approximately 0.75).

TABLE 2
The 15 most differentially methylated loci in the comparison of buccal
and WB DNA chromosomal localization of the CpG residue targeted
by the Illumina probe is given by its genome build 37 position

| | | Base Pair | Absolute |
|---|---|---|---|
| Chromosome | Probe ID | (Build 37) | Differential Methylation |
| 11 | cg25574765 | 70211531 | 0.91 |
| 20 | cg03841065 | 35274627 | 0.91 |
| 11 | cg10511890 | 47416487 | 0.90 |
| 12 | cg08075204 | 51718112 | 0.90 |
| 7 | cg24620436 | 4918906 | 0.90 |
| 20 | cg07598052 | 35274639 | 0.90 |
| 16 | cg04921315 | 4000587 | 0.89 |
| 11 | cg26427109 | 60739019 | 0.89 |
| 2 | cg00438740 | 233924930 | 0.89 |
| 6 | cg09344348 | 170581085 | 0.89 |
| 11 | cg08141395 | 95987382 | 0.89 |
| 10 | cg24681845 | 126069759 | 0.89 |
| 19 | cg22824635 | 13112283 | 0.89 |
| 4 | cg14516100 | 186560083 | 0.89 |
| 1 | cg20820767 | 45082840 | 0.89 |

The difference between whole blood and saliva DNA methylation in the 15 paired samples was determined. Overall, the absolute average difference between the average methylation of the whole blood sample as compared to the saliva samples at the 848,525 sites covered by the Epic array was only 2.4%. This is not unexpected for two reasons. First, prior studies and those of others have shown that, on average, 70% of DNA in saliva comes from whole blood, with the remainder coming from buccal cells. Therefore, generally, saliva samples look more like whole blood than they do buccal samples. Second, genome wide methylation is profoundly bimodal, with most loci being either completely methylated or completed unmethylated. Therefore, these paired whole blood/saliva samples are more similar to one another than those of pure whole blood and pure buccal cells.

However, the methylation sites that are most interesting to biologists are not those that are always completely methylated or demethylated. Rather, the most interesting are those whose methylation status can vary as a function of environmental exposure, such as seen in epigenetic aging, alcohol consumption or smoking. By and large, these loci are not hypermethylated and their set point varies between tissues. For example, methylation of the cg05575921 locus is 64% in Lowe et al.'s buccal cell data (Epigenetics, 8:445-54), yet 84% in the blood from non-smokers. As noted in the initial example with the alcohol loci, compensation for the differences in the set points of the four loci in the alcohol marker improves prediction. Not surprisingly, all of those four loci fall in the midrange of methylation (Philibert et al., 2019, J. Ins. Med., 48(1):90-102).

### Example 5—The DMR16 Correction Works at Many Loci

Although any locus that has a substantial difference in methylation (>1%) could benefit from heterogeneity correction, loci with the greatest differences in tissue set points (defined as the amount of methylation at a particular loci in a particular cell or tissue in individuals without disease or exposure will benefit the most. To get an understanding of the likelihood of this benefit, the Epic methylation data from 15 paired samples was first analyzed to identify those loci from the set of markers with a difference of greater than 5% methylation between saliva and whole blood. Overall, 49,982 of the 848,525 probes had an average difference of greater than 5% between the two sources of DNA. So that the effects of the admixture correction would be more apparent, those data were filtered to focus on those data whose set points in whole blood were least affected by probe measurement issues (non-specific probes or genetic confounding) or by uncontrolled environmental exposures. In other words, the loci whose methylation values were relatively invariant from sample to sample in whole blood, but not in saliva, were selected. Finally, those loci were filtered to include only those whose values are given in both the Epic and the 450K arrays (total n=399, 470).

Using the information from cg02614661, the CpG locus next to the locus assayed by the DMR16 assay, the saliva DNA methylation value was corrected for each sample for the top 15 loci identified above (i.e., cg06760305, cg25940946, cg10952220, cg09614653, cg20303441, cg01778994, cg07768107, cg13981380, cg02935132, cg16440978, cg15844596, cg22029597, cg12504877, cg07274406 and cg12086464) to see if compensating for cellular heterogeneity improved the correlation between the blood and the saliva samples. To do this, the proportion of the DNA in saliva arising from blood cells was first calculated using the formula adapted from the prior studies of DMR16:

Observed DMR16(saliva)=0.97X+0.18(1−X)

where Observed(saliva) is the amount of DNA methylation in saliva at DMR16, X is the proportion of DNA in saliva originating from whole blood, (1−X) is the proportion of DNA in the saliva originating from buccal cells, 0.97 is the fractional methylation of the CpG immediately adjacent to cg02614661 in whole blood (from the array data), and 0.18 is the fractional methylation of the CpG immediately adjacent to cg02614661 in buccal cell DNA (from the data set in Lowe et al., 2013, Epigenetics, 8(4):445-54).

For each sample, the imputed value of “X” was multiplied by the average DNA methylation value for each locus in whole blood, “(1−X)” was multiplied by the average DNA methylation value for each locus in buccal cells, and these values were added together.

It was then determined whether the predicted methylation value correlated better with the observed value in saliva than with the observed value in the matched whole blood sample. Overall, this correction improved the average correlation at these 15 loci by an R² of 0.06 (i.e. 6%), which demonstrated that the DMR16 cell correction can be used at any locus that has a variable set point.

But what does one do if the methylation set point of each tissue is not fixed, but varies as a function of exposures, such as smoking? As was observed in 2015, even if the tissue set points are different, the changes in methylation are in the same direction and their magnitude is very similar (Teschendorff et al., 2015, JAMA Oncol., 1:476-85). For example, at cg05575921, the change in methylation per 10-pack-year of smoking (was −5.5% for buccal cells and −5% for blood (i.e., methylation decreases the more one smokes). So, to determine smoking status for a given saliva sample, cg05575921 methylation in the saliva sample is determined, then the relative contribution of whole blood DNA (X) and buccal DNA (1−X) to the sample is determined using the information from the DMR16 assay. Then, the best fit of the below formula is determined by starting with the default/no exposure values of cg05575921 in whole blood (Q) and buccal (R) of 0.84 and 0.7, respectively. 0.01 is subtracted from Q (0.84) and R (0.7) simultaneously and iteratively (start with 0.84 and 0.7; then 0.83 and 0.69; then 0.82 and 0.68, etc.) until the resulting value of the formula best matches the Observed cg05575921 in the saliva. Alternatively, one can just solve the formula algebraically to come to an exact result.

Observed DMR16(saliva)=QX+R(1−X)

That best fitting pair of values is the set of whole blood and buccal cell DNA methylation levels that contributed to the saliva. Because blood DNA is the most common biomaterial used in medical methylation studies (e.g., smoking), the resulting imputed blood DNA value then can be used to impute smoking status. Alternatively, this formula can be used to determine the DNA methylation for any locus that varies in whole blood and buccal cell as a function of illness or environmental exposure.

### Example 6—Generalizability of the DMR16 Approach to Use Methylation at Other Loci to Determine Cell Proportion

It would be understood that any CpG locus that demonstrates substantial differential methylation between whole blood and buccal DNA can be used to impute the mix of buccal and whole blood contributions to saliva DNA. Indeed, the cg02614661 locus, right next to where the DMR16 locus is based, is only the 4744^{th} highest ranked site in the survey. Since there are 28 million CpG sites in the human genome, and the arrays only measure a fraction of these sites, it is likely that there are many sites that can be used in this correction scheme. For example, since the differential methylation for each of the loci in Table 1 is greater than that for cg02614661 (i.e., the DMR16 locus), each should have excellent capacity to correct for cellular heterogeneity. This was tested using the formula described above and substituting the array data with respect to the buccal and whole blood DNA methylation set points for the top two loci from Table 1, cg25574765 and cg03841065, for that of cg02614661 (DMR16) to adjust for cellular heterogeneity. Using this approach, the average correlation of methylation values from the whole blood and the saliva samples at the 15 loci from the above example improved by 7% and 8%, respectively.

Any of these regions can be used in digital PCR or sequencing based approaches similar to what was done with DMR16. It would be appreciated that the 15 CpG regions interrogated tends to be CpG rich, often with confounding local genetic variation. One particular example from Table 1 is cg08141395, which only has one other CpG residue within 60 bp of the targeted CpG site. Similar to the above two sites, inserting its methylation values from the array into the heterogeneity correction improved the average correlation of methylation values from the whole blood and the saliva samples at the 15 loci by nearly 8%. The lack of confounding CpG sites and genetic variation makes cg08141395 an outstanding candidate for use in a digital PCR assay.

To show that an assay can be constructed for this or another locus using the information from the Epic annotation file (which is available at support.Illumina.com/ on the World Wide Web) and the UCSC genome browser (Kent et al., 2002, Gen. Res., 12:996-1006), the sequence surrounding cg08141395 (DMR11), which corresponds to the sequence from Build 38 of the human genome (i.e., Chr11:96254008-96254429), was downloaded, and a methylation-sensitive digital PCR assay was designed. The targeted CpG residue is highlighted in grey in the sequence below, and the bisulfite converted sequence corresponding to that position is shown below the native sequence. The sequences of the outer (F1 and R1) and inner primers (R2 and F2) are single and double underlined, respectively, and the area targeted by the fluorescent, locked nucleic acid containing probes is boxed. Primers R1 and F1 were used for pre-amplification at 60° C. and primers R3 and F2 were used for amplification at 55° C.

| DMR11 Sequence | | |
|---|---|---|
| (SEQ ID NO: 11) | | |
| ATACACTGAAGGTATCACTTACACTTTCTTTAAAGGTAAGAATTTGTGAGACTTCTGGGAGA | | |
| ATTTTGACAGGTCCTATTAGAGGTATTTTAAAACACACAGGGGAAAGTGATTTGATGTTAAG | | |
| CAGTGGCAAATCTACACAAAAACAAAAACAGTCATCGGAGACTTTCACTCAATACAAAGTTC | | |
| 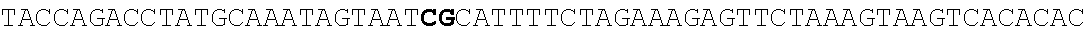 | | |
| ACAAACCTCAGTAGTAGCACAAAACATCCTTTGTTGCCGGACGTGAGAAAAACACACTCGCT | | |
| TCTAAAAAAAGCCATAGGAAGGAAGTGGAAGAACCTCAGGGGCGAGTGGGAGTGCGAAAGGA | | |
| ATGTTGCAGCTCTTTTTTTTTTTTTTTTTGAACATGTAAGCTTGCTGTGGTTATAGTAAGTT | | |
| TATATGTTTAAAAAAAAAAAAAAAAAGAGTTGTAATATTTTTTTXGTATTTTTATTXGTTTT | | |
| TGAGGTTTTTTTATTTTTTTTTTATGGTTTTTTTTAGAAGXGAGTGTGTTTTTTTTAXGTTX | | |
| 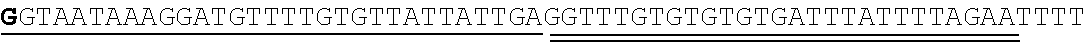 | | |
| 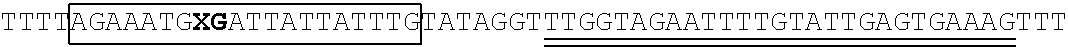 | | |
| 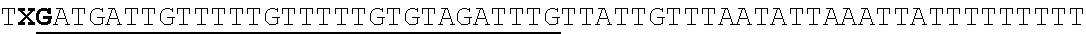 | | |
| GTGTGTTTTAAAATATTTTTAATAGGATTTGTTAAAATTTTTTTAGAAGTTTTATAAATTTT | | |
| TATTTTTAAAGAAAGTGTAAGTGATATTTTTAGTGTAT | | |
| DMR11 F1 | GGTAATAAAGGATGTTTTGTGTTATTATTGA | (SEQ ID NO: 12) |
| DMR11 F2 | GGTTTGTGTGTGTGATTTATTTTAG | (SEQ ID NO: 13) |
| DMR11 R2 | CTTTCACTCAATACAAAATTCTACCAA | (SEQ ID NO: 14) |
| DMR11 R1 | CAAATCTACACAAAAACAAAAACAATCATC | (SEQ ID NO: 15) |
| Methylated allele probe | A + TAA + T + CG + CATTT + T + CT | (SEQ ID NO: 16) |
| Unmethylated allele probe | ACAA + TAAT + C + A + CATTT + T + CT | (SEQ ID NO: 17) |

(+N corresponds to LNA residue)

Using this assay, DNA was amplified from four whole blood samples and the report of low average methylation of the locus in whole blood (0.6% by digital PCR; Lowe et al., 2013, Epigenetics, 8(4):445-54) was confirmed because the locus is almost completely methylated in buccal cells. This assay, like the DMR16 assay, worked to correct for the effects of cellular heterogeneity in saliva DNA samples.

As Table 3 shows, the DMR11 correction allow us to determine the methylation in the whole blood constituent of saliva DNA which enables diagnostics metrics developed for whole blood DNA to be used in conjunction with saliva DNA.

TABLE 3
Application of the DMR11 Assay to Adjust for Methylation
within AHRR using Both Saliva and Blood DNA

| Sample | | 95% | | 95% | % | True blood |
|---|---|---|---|---|---|---|
| ID | cg05575921 | CI | DMR11 | CI | WBC | methylation |
| 1 | 70.4 | 1.1 | 13.66 | 0.79 | 87 | 72 |
| 2 | 59.4 | 2.2 | 30.6 | 2.1 | 69 | 64 |
| 3 | 64.7 | 1.3 | 20 | 1 | 80 | 68 |
| 4 | 51.1 | 1.8 | 19.4 | 1.2 | 81 | 54 |
| 5 | 54.5 | 1.3 | 40.3 | 1.3 | 59 | 61 |
| 6 | 23.4 | 1.3 | 10.82 | 0.94 | 90 | 25 |
| 7 | 30.1 | 1.6 | 25.5 | 1.6 | 74 | 34 |
| 8 | 73.5 | 1.2 | 18.5 | 1 | 82 | 76 |
| 9 | 30.2 | 1.2 | 38.2 | 1.3 | 61 | 36 |
| 10 | 14.97 | 0.8 | 20.09 | 0.92 | 80 | 18 |
| 11 | 29.1 | 1.3 | 6.19 | 0.55 | 95 | 30 |
| 12 | 58 | 1.3 | 5.92 | 0.46 | 95 | 59 |
| 13 | 29.5 | 2.1 | 82.7 | 1.7 | 15 | 42 |
| 14 | 24.8 | 1.3 | 16.47 | 0.81 | 84 | 27 |
| 15 | 19.5 | 1.1 | 7.12 | 0.51 | 94 | 20 |
| 16 | 31.7 | 2 | 62.4 | 2.1 | 36 | 41 |
| 17 | 37.6 | 2.8 | 80.5 | 2.4 | 17 | 50 |
| 18 | 63.3 | 1.3 | 7.84 | 0.6 | 93 | 64 |
| 19 | 58.4 | 1.4 | 14.92 | 0.83 | 86 | 61 |
| 20 | 24 | 1.2 | 75.6 | 1.2 | 22 | 36 |
| 21 | 73.9 | 3.7 | 20.2 | 3.2 | 80 | 77 |
| 22 | 31.4 | 1.8 | 86.4 | 1.4 | 11 | 45 |

### Example 7—Summary

These experiments demonstrated that: 1) the human methylome contains a large number of sites whose methylation is markedly different in buccal DNA as compared to whole blood DNA, 2) the method of using information from the DMR16 (near cg02614661) locus can correct for admixture in saliva DNA and allow imputation of the methylation values of the buccal and whole blood DNA contribution in a saliva sample, 3) the general principle outlined at the DMR16 locus (near cg02614661) can be harnessed and applied to a number of other loci, and 4) methylation status at these other loci also can be assessed using affordable PCR or sequencing technologies.

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

1. A method of correcting for cellular heterogeneity in an oropharyngeal biological sample used to determine the methylation status of a target nucleic acid sequence, the method comprising:
providing the oropharyngeal biological sample, the oropharyngeal biological sample comprising buccal cells and white blood cells;
determining the methylation status of the target sequence and at least one differentially methylated region (DMR) loci in the biological sample;
applying a formula to the methylation status of the target sequence and the at least one DMR loci in the biological sample to determine an amount of white blood cells and an amount of buccal cells in the biological sample; and
correcting for cellular heterogeneity in the biological sample when determining the DNA methylation status of the target sequence.

2. The method of claim 1, wherein the oropharyngeal biological sample is saliva or sputum.

3. The method of claim 1, wherein the absolute difference between the methylation status at the DMR loci in whole blood and at the DMR loci in buccal cells is at least 0.5.

4. The method of claim 3, wherein the absolute difference between the methylation status at the DMR loci in whole blood and the DMR loci in buccal cells is at least 0.8.

5. The method of claim 3, wherein the absolute difference between the methylation status at the DMR loci in whole blood and the DMR loci in buccal cells is at least 0.9.

6. The method of claim 1, wherein the DMR loci is selected from DMR11 (cg25574765), DMR20 (cg03841065), DMR11 (cg10511890), DMR12 (cg08075204), DMR7 (cg24620436), DMR20 (cg07598052), DMR16 (cg04921315), DMR11 (cg26427109), DMR2 (cg00438740), DMR6 (cg09344348), DMR11 (cg08141395), DMR10 (cg24681845), DMR19 (cg22824635), DMR4 (cg14516100), and DMR1 (cg20820767).

7. The method of claim 1, wherein the DMR loci is DMR16 and the formula comprises
DMR16(obs)=(0.97X+0.18(1−X))
wherein DMR16(cg05575921)(obs) is the observed methylation signal in the heterogeneous biological sample; and X is the white blood cell contribution to the biological sample.

8. The method of claim 1, wherein the DMR loci is DMR11(cg08141395) and the formula comprises
DMR11(obs)=(0.01X+0.99(1−X))
wherein DMR11(obs) is the observed methylation signal in the heterogeneous biological sample; and X is the white blood cell contribution to the biological sample.

9. The method of claim 1, wherein the determining step comprises PCR and/or sequencing.

10. A method of correcting for cellular heterogeneity in a biological sample, comprising:
(a) providing a heterogeneous biological sample comprising buccal cells and white blood cells;
(b) contacting nucleic acid from the biological sample with bisulfite under alkaline conditions;
(c) performing methylation-sensitive PCR on the bisulfite-converted nucleic acid with a pair of primers that amplifies a first locus comprising at least one target CpG dinucleotide and a pair of primers that amplifies at least one DMR loci;
(d) determining the methylation status of the at least one target CpG dinucleotide and the methylation status of the at least one DMR loci; and
(e) correcting for cellular heterogeneity in the biological sample using a pre-determined formula.

11. The method of claim 10, wherein the absolute difference between the methylation status at the DMR loci in whole blood and the DMR loci in buccal cells is at least 0.5.

12. The method of claim 11, wherein the absolute difference between the methylation status at the DMR loci in whole blood and the DMR loci in buccal cells is at least 0.8.

13. The method of claim 11, wherein the absolute difference between the methylation status at the DMR loci in whole blood and the DMR loci in buccal cells is at least 0.9.

14. The method of claim 10, wherein the DMR is selected from DMR11 (cg25574765), DMR20 (cg03841065), DMR11 (cg10511890), DMR12 (cg08075204), DMR7 (cg24620436), DMR20 (cg07598052), DMR16 (cg04921315), DMR11 (cg26427109), DMR2 (cg00438740), DMR6 (cg09344348), DMR11 (cg08141395), DMR10 (cg24681845), DMR19 (cg22824635), DMR4 (cg14516100), and DMR1 (cg20820767).

15. The method of claim 10, wherein the DMR loci is DMR16 and the predetermined formula comprises
DMR16(obs)=(0.97X+0.18(1−X))
wherein DMR16(obs) is the observed methylation signal in the biological sample; and X is the white blood cell contribution to the biological sample.

16. The method of claim 10, wherein the DMR loci is DMR11 and the predetermined formula comprises
DMR11(obs)=(0.01X+0.99(1−X))
wherein DMR11(obs) is the observed methylation signal in the biological sample; and X is the white blood cell contribution to the biological sample.

17. The method of claim 10, wherein the determining step further comprises sequencing.

18. A method for identifying a differentially methylated region (DMR) loci that can be used to correct for cellular heterogeneity in a biological sample, comprising:
(a) comparing the methylation status of a plurality of loci in a first component of the heterogeneous biological sample and the methylation status of a plurality of loci in a second component of the heterogeneous biological sample;
(b) identifying one or more loci from the plurality of loci that are differentially methylated in the first component of the heterogeneous biological sample relative to the second component of the heterogeneous biological sample, wherein the absolute difference between the methylation status in the first component and the methylation status in the second component of the one or more identified loci is at least 0.5,
thereby identifying a DMR loci that can be used to correct for cellular heterogeneity in a biological sample.

19. The method of claim 18, the absolute difference between the methylation status in the first component and the methylation status in the second component is at least 0.8.

20. The method of claim 18, the absolute difference between the methylation status in the first component and the methylation status in the second component is at least 0.9.

21. The method of claim 18, wherein the DMR is selected from DMR11 (cg25574765), DMR20 (cg03841065), DMR11 (cg10511890), DMR12 (cg08075204), DMR7 (cg24620436), DMR20 (cg07598052), DMR16 (cg04921315), DMR11 (cg26427109), DMR2 (cg00438740), DMR6 (cg09344348), DMR11 (cg08141395), DMR10 (cg24681845), DMR19 (cg22824635), DMR4 (cg14516100), and DMR1 (cg20820767).

22. An article of manufacture to correct for cellular heterogeneity in a biological sample when determining the nucleic acid methylation status of a target sequence in the biological sample, comprising:
a first pair of DMR primers; and
at least one DMR probe that detects either a methylated or an unmethylated CpG dinucleotide.

23. The article of manufacture of claim 22, further comprising a second pair of DMR primers.

24. The article of manufacture of claim 22, comprising:
a first pair of DMR11 primers; and
at least one DMR11 probe that detects either a methylated or an unmethylated CpG dinucleotide.

25. The article of manufacture of claim 24, wherein the first pair of DMR11 primers comprises a first member and a second member, wherein the first member has the sequence shown in SEQ ID NO:12 and the second member has the sequence shown in SEQ ID NO:15.

26. The article of manufacture of claim 24, wherein the at least one DMR11 probe is selected from the sequence shown in SEQ ID NO:16 and the sequence shown in SEQ ID NO:17.

27. The article of manufacture of claim 24, further comprising a second pair of DMR11 primers.

28. The article of manufacture of claim 27, wherein the second pair of DMR11 primers comprises a first member and a second member, wherein the first member has the sequence shown in SEQ ID NO:13 and the second member has the sequence shown in SEQ ID NO:14.

29. The article of manufacture of claim 22, comprising:
a first pair of DMR16 primers; and
at least one DMR16 probe that detects either a methylated or an unmethylated CpG dinucleotide.

30. The article of manufacture of claim 29, wherein the first pair of DMR16 primers comprises a first member and a second member, wherein the first member has the sequence shown in SEQ ID NO:3 and the second member has the sequence shown in SEQ ID NO:5.

31. The article of manufacture of claim 29, wherein the at least one DMR16 probe is selected from the sequence shown in SEQ ID NO:7 and the sequence shown in SEQ ID NO:8.

32. The article of manufacture of claim 29, further comprising a second pair of DMR16 primers.

33. The article of manufacture of claim 32, wherein the second pair of DMR16 primers comprises a first member and a second member, wherein the first member has the sequence shown in SEQ ID NO:4 and the second member has the sequence shown in SEQ ID NO:6.

34. The article of manufacture of claim 22, wherein at least one member of the first pair of primers, at least one member of the second pair of primers, or the at least one probe comprises a modified nucleotide.

35. The article of manufacture of claim 22, further comprising reagents for bisulfite converting nucleic acid.

36. The article of manufacture of claim 22, further comprising reagents for amplifying nucleic acid.

37. The article of manufacture of claim 22, further comprising at least one probe that detects either the methylated or the unmethylated CpG dinucleotide.

38. The article of manufacture of claim 22, further comprising a minor groove binder (MGB).


SEQUENCE LISTING (17 sequences)

SEQ ID NO: 1 - DNA, 1060 bp, Homo sapiens
ttgtccctaagaggcatcttcctcaggggctggtggagctgccatgaaagcaaacgcacagccaaaccccgggtggggggaaggcaaactgcaaacgccgcggcgaccccggcacagcagccctgtcagcaggattcccccgagagcggggtaattgcggtgggaacgagcgctccaaaggccctggggagatgatttcagggaaaagtggccttgatccctgagtcaggcagatgcggccatgggaaccatccaccccgaggctggaggggagactccgccggtggctaaagccatcctgctgacggggcccagggacgcccccagtggccaaacgcacgtgggaacgggatcttccccctcctctgtgatgcggccaaccctccaagcctctggctcctgactcagaggacgatgtctccccatgaacgcagtgtccctggagagaagagcctgcccagcgtggggaacatgggaatgtggagatggagggcatcccggaacctcagggctgacgggcccctgccccagccctggaaacacctcagggacaagagagtcactcctcaagcgcggactttccactgtgctggggccttccgccttccaaccactctggccccttggggctctaggtggagtgtgctgaacagtgtccccaaaattcacgtccagtggagctgcgggatgtgtcctagctgcaaatgcggtctccgcaggtgcaattagcgaaggaccttgagatgagatcatcccggattagagtaggcactaaggccgacgacaagtgtcctcagcgacacagagacagatccaggggagacagagccagagccagcccaggatgcctggagccacgggcagctggaaggggaaggtggcaccttgggtctggacctctggctgccaggaccaggaccgtctgcatgtctgttgctaaagccagtctgtaggcatctgtcaccacacatgaggggccaaccgtgccacccaggggagacctcccggcttctcagtgcatcaggcatggtcaggggcaggacagctgcagctcacaccc

SEQ ID NO: 2 - DNA, 1060 bp, Artificial
  chemically-generated sequence
ttgtttttaagaggtattttttttaggggttggtggagttgttatgaaagtaaacgtatagttaaatttcgggtggggggaaggtaaattgtaaacgtcgcggcgatttcggtatagtagttttgttagtaggattttttcgagagcggggtaattgcggtgggaacgagcgttttaaaggttttggggagatgattttagggaaaagtggttttgatttttgagttaggtagatgcggttatgggaattatttatttcgaggttggaggggagatttcgtcggtggttaaagttattttgttgacggggtttagggacgtttttagtggttaaacgtacgtgggaacgggatttttttttttttttgtgatgcggttaattttttaagtttttggtttttgatttagaggacgatgtttttttatgaacgtagtgtttttggagagaagagtttgtttagcgtggggaatatgggaatgtggagatggagggtatttcggaattttagggttgacgggtttttgttttagttttggaaatattttagggataagagagttattttttaagcgcggattttttattgtgttggggtttttcgttttttaattattttggttttttggggttttaggtggagtgtgttgaatagtgtttttaaaatttacgtttagtggagttgcgggatgtgttttagttgtaaatgcggttttcgtaggtgtaattagcgaaggattttgagatgagattatttcggattagagtaggtattaaggtcgacgataagtgtttttagcgatatagagatagatttaggggagatagagttagagttagtttaggatgtttggagttacgggtagttggaaggggaaggtggtattttgggtttggatttttggttgttaggattaggatcgtttgtatgtttgttgttaaagttagtttgtaggtatttgttattatatatgaggggttaatcgtgttatttaggggagatttttcggttttttagtgtattaggtatggttaggggtaggatagttgtagtttatattt

SEQ ID NO: 3 - DNA, 22 bp, Artificial
  Synthetic oligonucleotide
gtagtgtttttggagagaagag

SEQ ID NO: 4 - DNA, 19 bp, Artificial
  Synthetic oligonucleotide
tatgggaatgtggagatgg

SEQ ID NO: 5 - DNA, 19 bp, Artificial
  Synthetic oligonucleotide
cacactccacctaaaaccc

SEQ ID NO: 6 - DNA, 25 bp, Artificial
  Synthetic oligonucleotide
ctctcttatccctaaaatatttcca

SEQ ID NO: 7 - DNA, 20 bp, Artificial
  Synthetic oligonucleotide
tagggttgatgggtttttgt

SEQ ID NO: 8 - DNA, 20 bp, Artificial
  Synthetic oligonucleotide
tagggttgacgggtttttgt

SEQ ID NO: 9 - DNA, 11 bp, Artificial
  Synthetic oligonucleotide
ttgacgggttt

SEQ ID NO: 10 - DNA, 11 bp, Artificial
  Synthetic oligonucleotide
ttgatgggttt

SEQ ID NO: 11 - DNA, 844 bp, Homo sapiens
  misc_feature (479)..(479): n = C or U
  misc_feature (491)..(491): Xaa = C or U
  misc_feature (537)..(537): n = C or U
  misc_feature (554)..(554): n = C or U
  misc_feature (558)..(558): n = C or U
  misc_feature (633)..(633): n = C or U
  misc_feature (684)..(684): n = C or U
atacactgaaggtatcacttacactttctttaaaggtaagaatttgtgagacttctgggagaattttgacaggtcctattagaggtattttaaaacacacaggggaaagtgatttgatgttaagcagtggcaaatctacacaaaaacaaaaacagtcatcggagactttcactcaatacaaagttctaccagacctatgcaaatagtaatcgcattttctagaaagagttctaaagtaagtcacacacacaaacctcagtagtagcacaaaacatcctttgttgccggacgtgagaaaaacacactcgcttctaaaaaaagccataggaaggaagtggaagaacctcaggggcgagtgggagtgcgaaaggaatgttgcagctctttttttttttttttttgaacatgtaagcttgctgtggttatagtaagtttatatgtttaaaaaaaaaaaaaaaaagagttgtaatatttttttngtatttttattngtttttgaggtttttttattttttttttatggttttttttagaagngagtgtgttttttttangttnggtaataaaggatgttttgtgttattattgaggtttgtgtgtgtgatttattttagaattttttttagaaaatgngattattatttgtataggtttggtagaattttgtattgagtgaaagttttngatgattgtttttgtttttgtgtagatttgttattgtttaatattaaattatttttttttgtgtgttttaaaatatttttaataggatttgttaaaatttttttagaagttttataaatttttatttttaaagaaagtgtaagtgatatttttagtgtat

SEQ ID NO: 12 - DNA, 31 bp, Artificial
  Synthetic oligonucleotides
ggtaataaaggatgttttgtgttattattga

SEQ ID NO: 13 - DNA, 25 bp, Artificial
  Synthetic oligonucleotides
ggtttgtgtgtgtgatttattttag

SEQ ID NO: 14 - DNA, 27 bp, Artificial
  Synthetic oligonucleotides
ctttcactcaatacaaaattctaccaa

SEQ ID NO: 15 - DNA, 30 bp, Artificial
  Synthetic oligonucleotides
caaatctacacaaaaacaaaaacaatcatc

SEQ ID NO: 16 - DNA, 15 bp, Artificial
  Synthetic oligonucleotides
ataatcgcattttct

SEQ ID NO: 17 - DNA, 18 bp, Artificial
  Synthetic oligonucleotides
acaataatcacattttct